(19) United States
(12) Patent Application Publication
(10) Pub. No.: US 2025/0356976 A1
(43) Pub. Date: Nov. 20, 2025

(54) DETERMINING AND OPTIMIZING INDIVIDUALIZED TREATMENT REGIMENS

(71) Applicant: Noho Allergy, Inc, New York, NY (US)

(72) Inventors: Shyam Joshi, Lake Oswego, OR (US); Alimohammad Shahabi Sirjani, Cottonwood Heights, UT (US); Benjamin Joseph Oyler, Riverton, UT (US)

(21) Appl. No.: 18/871,406

(22) PCT Filed: Jun. 5, 2023
(86) PCT No.: PCT/US2023/024439

§ 371 Date: Dec. 3, 2024

Related U.S. Application Data
(60) Provisional application No. 63/348,809, filed on Jun. 3, 2022.

(51) Int. Cl. G16H 20/10; G16H 80/00

(52) U.S. Cl. CPC G16H 20/10; G16H 80/00

(57) ABSTRACT

A method and system may be provided to identify allergen triggers and formulate custom allergy treatment regimens based on patient information and test results.

### FIELD

The present invention relates generally to identification of allergen triggers and the formulation of custom allergy treatment regimens based on patient information and test results.

### BACKGROUND

Traditionally, allergy sufferers have relied upon antihistamines or other medications to treat their symptoms. Allergy medication generally only treats the symptoms and not the underlying allergy itself.

Immunotherapy has been used to reduce the reaction a patient has to a particular allergen, but requires regular office visits to an allergist for allergy testing and to receive subcutaneous immunotherapy (SCIT) known as allergy shots.

### SUMMARY

The systems and methods described herein provide for a sublingual immunotherapy (SLIT) treatment determination and optimization framework. The framework may be used to create custom and individualized treatment regimens for patients. In some embodiments, a patient may be mailed a testing kit in response to a prospective patient request, physicians request on behalf of a patient or other indication of interest in starting a SLIT regimen to treat one or more allergies. The patient may use the testing kit to collect one or more specimens, such as blood. The patient may then mail or deliver the specimen to a receiving facility, laboratory or physician's office for processing.

In some embodiments the processing is configured to test a patient with regard to the plurality of allergens and generating a specific IgE value for each allergen. The test results may then be transmitted or otherwise provided from the testing facility to the patient, the patient's physician and a server configured to analyze the results.

In some embodiments, the framework may be configured to collect user input from one or more client devices, wherein the user input comprises a questionnaire or survey. The system may then further receive, by a prescription logic module operating on the server, one or more regional pollination schedules, one or more allergen extract mapping tables and one or more cross-reactive allergen tables. The retrieved information may be based at least in part on the user input, the test results and a location of the user.

In some embodiments, the system may be configured to determine whether a patient is qualified to receive SLIT treatments. A qualification protocol may be followed to make the qualification determination.

In some embodiments, the system may be configured to identify, by an analysis module, one or more target allergens that a qualified patient is to be treated for. The analysis may comprise determining a correlation between the user input and the test results, wherein the test results comprise IgE levels of each allergen tested. The system may then generate a list of allergies with a specific IgE value above a first predetermined threshold. The system may then generate a ranked list of one or more allergy triggers, wherein the allergy triggers and their ranking in the ranked list are determined based on the user input, the month of trigger for the allergy trigger and a dataset of regional allergens and month values for the location of the user.

In some embodiments, a treatment formulation for the patient may be generated by a dosing module. The dosing module may be configured to determine a maximum number of extracts to prescribe the patient, or receive a predetermined parameter indicating the maximum number of extracts that may be prescribed to a patient. The system may select, from the ranked list of allergy triggers, a first target allergy trigger, wherein the selecting is based at least partly on the month of trigger for the allergy triggers, the IgE levels for corresponding allergens and regional allergens present during the month of trigger. The first allergy trigger may then be added to the treatment formulation.

In some embodiments, the system may further be configured to determine, by the analysis module, a level of cross-reactivity between the allergy triggers in the ranked list of allergy triggers and select, from the ranked list of allergy triggers, a second target allergy trigger, wherein the selecting is based at least partly on the month of trigger for the allergy triggers, the IgE levels for corresponding allergens, regional allergens present during the month of trigger and the level of cross-reactivity between the first target allergy trigger and the second target allergy trigger. When the cross-reactivity level between the first target allergy trigger and the second target allergy trigger is below a second predetermined threshold and the IgE value for the second target allergy trigger is above the first predetermined threshold the second allergy trigger may be added to the treatment formulation

In some embodiments, the system may then map the target allergy triggers in the treatment formulation to one or more extracts and selecting one or more extracts to be compounded into the treatment formulation. The system may then be configured to determine a concentration and volume for each selected extract, wherein the concentration and volume are based at least in part on the user input and IgE levels of the allergy triggers corresponding to the extracts.

In some embodiments, the system may further be configured to determine a dosing schedule for the patient. Compounding information for the treatment formulation may be transferred to a compounding pharmacy to be compounded into a custom SLIT treatment and mailed or otherwise provided to the patient. The SLIT treatment may be included in kit form, along with instructions for administration and the determined dosing schedule.

The features and components of these embodiments will be described in further detail in the description which follows. Additional features and advantages will also be set forth in the description which follows, and in part will be implicit from the description, or may be learned by the practice of the embodiments. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1 is a diagram illustrating an exemplary framework in which some embodiments may operate.

FIG. 2 is a diagram illustrating an exemplary server in accordance with aspects of the present disclosure.

FIG. 3A is a flow chart illustrating an exemplary method that may be performed in accordance with some embodiments.

FIG. 3B is a flow chart illustrating an exemplary method that may be performed in accordance with some embodiments.

FIG. 3C is a flow chart illustrating an exemplary method that may be performed in accordance with some embodiments.

FIG. 3D is a flow chart illustrating an exemplary method that may be performed in accordance with some embodiments.

FIG. 4A is an exemplary allergy test result in accordance with some embodiments.

FIG. 4B is an exemplary allergy test result in accordance with some embodiments.

FIG. 4C is an exemplary regional allergen pollination schedule in accordance with some embodiments.

FIG. 4D is an exemplary regional allergen pollination schedule in accordance with some embodiments.

FIG. 4E is an exemplary patient allergy history in accordance with some embodiments.

FIG. 4F is an exemplary ranked list of allergens in accordance with some embodiments.

FIG. 4G is an exemplary regional allergen pollination schedule in accordance with some embodiments.

FIG. 4H is an exemplary patient allergy history in accordance with some embodiments.

FIG. 4I is an exemplary ranked list of allergens in accordance with some embodiments.

FIG. 4J is an exemplary allergy test result in accordance with some embodiments.

FIG. 4K is an exemplary allergy test result in accordance with some embodiments.

FIG. 4L is an exemplary regional allergen pollination schedule in accordance with some embodiments.

FIG. 4M is an exemplary patient allergy history in accordance with some embodiments.

FIG. 4N is an exemplary ranked list of allergens in accordance with some embodiments.

FIG. 5 is an exemplary framework diagram in accordance with some embodiments.

FIG. 6A is an exemplary user interface in accordance with some embodiments.

FIG. 6B is an exemplary user interface in accordance with some embodiments.

FIG. 6C is an exemplary user interface in accordance with some embodiments.

FIG. 6D is an exemplary user interface in accordance with some embodiments.

FIG. 6E is an exemplary user interface in accordance with some embodiments.

FIG. 6F is an exemplary user interface in accordance with some embodiments.

FIG. 6G is an exemplary user interface in accordance with some embodiments.

FIG. 6H is an exemplary user interface in accordance with some embodiments.

FIG. 6I is an exemplary user interface in accordance with some embodiments.

FIG. 6J is an exemplary user interface in accordance with some embodiments.

FIG. 6K is an exemplary user interface in accordance with some embodiments.

FIG. 6L is an exemplary user interface in accordance with some embodiments.

FIG. 7 is a diagram illustrating an exemplary computer/control system that may perform processing in some embodiments and in accordance with aspects of the present disclosure.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

The following generally relates to a system, platform and methods for determining and optimizing custom and individualized SLIT treatment regimens for patients. In some embodiments, prospective patients may initiate a qualification protocol by registering with a SLIT treatment service provider or by contacting a physician. When a prospective patient registers or otherwise initiates the qualification protocol with the SLIT treatment service provider, the provider may contact a physician to request an order for one or more allergy tests be generated. The patient may be sent a specimen collection kit in the mail. The kit may be used by the patient to collect a specimen, and the specimen may be mailed to a collection or testing facility to be processed. In some embodiments, the patient may visit a facility to have their specimen collected for testing. A patient may also bring a specimen collected at home to a facility for testing to be performed.

In some embodiments, patients may be required to have a physician authorization for the test before the testing of a specimen may begin. The collected sample may then be tested against a plurality of allergens to determine specific IgE levels for each allergen. In some embodiments there may be a predetermined IgE level threshold, above which, the allergen would be identified as a candidate for treatment. In some embodiments, a ranked list of allergens may be created for some or all allergens that exceed the IgE level threshold. The ranking may be in order according to IgE level results.

In some embodiments, the physician may review the test results before approving the test results for analysis to determine formulation, dosage and scheduling of treatment.

In some embodiments, a prospective patient may be asked to answer a questionnaire to determine an allergy history for the patient. Information related to the symptoms, indoor vs outdoor allergy triggers, severity, season and time at which the patient experiences allergy symptoms may be collected. One or more months may be specified as problematic for outdoor allergies in which the symptoms are the most severe. The patient may also provide information related to weather and regions which may trigger their allergies.

Based on one or more regional pollination schedules, the IgE levels in the patients test results and personal history information collected from the patient, a qualification of the patient to participate in the SLIT treatment may be determined. In some embodiments, the one or more regional pollination schedules, the IgE levels in the patients test results and personal history information collected from the patient may be used to identify one or more allergens that are the most likely to be symptomatic for the patient and generate a prescription based on that. Based on the IgE levels and feedback from the patient, a formulation, dosage and schedule may be optimized for the patient's specific needs.

In some embodiments, diagnostics and therapeutics may be combined into a single platform where the analysis of test results, coordination of communication between patient and physician, optimization of treatment regimens and compounding of the treatment formulations are performed automatically and with minimal human interaction.

In some embodiments, during the course of treatment, additional tests may be performed and/or additional questionnaires may be required for patients to complete to determine the efficacy of the treatment and to determine any side effects related to the treatment. In some embodiments, the patient may be required to provide feedback at specified intervals to track the effects of the treatment over time. Adverse effects may be reported at any time. Reformulation and dosing schedule changes may be initiated based on the patient feedback and test results. Patient test results, questionnaire answers, feedback and other data collected with regard to the patient, the allergens being treated and the treatment formulation and dosage schedule may be stored and used to train one or more machine learning models to identify treatment modifications with a high probability of success for a specific patient.

In some embodiments, answers collected from patient questionnaires may trigger a process to collect a new sample for additional testing. A collection kit may be automatically dispatched to the patient and an order generated for testing to be performed at a laboratory based on feedback and input from the patient. Reformulation may then be performed based on the patient's recent questionnaire and feedback, the results from the additional testing and/or knowledge learned from the analysis of historic data for the patient as well as the knowledge learned from the analysis of other patients that have previously participated in the treatment program.

In some embodiments, one or more machine learning models may be trained on data collected from a plurality of patients that have been or are currently being treated. In some embodiments, there may be different machine learning models used for different categories of allergens, different demographics, and different regions. Models may be retrained based on receiving of adverse events or significant patient improvements.

In some embodiments, the allergens tested for and treated may be indoor, outdoor, food or allergens unique to specific work environments. Indoor allergens may include pet hair and dander, dust mites and mold. Outdoor allergens may include tree pollen, grass pollen and weed pollen.

In some embodiments, patient demographics, location, test results, questionnaire answers and personal history may be used to generate a patient profile. A patient profile may be analyzed as a whole by a machine learning model to determine a treatment formulation, dosage and schedule optimized to the patient's specific profile. As more information is collected from the patient, the patient profile may change or evolve over time.

Patient may provide information related to the severity of different allergens with relation to a uniform scale. Patients may also be asked to provide a relative comparison of severity between two or more allergens.

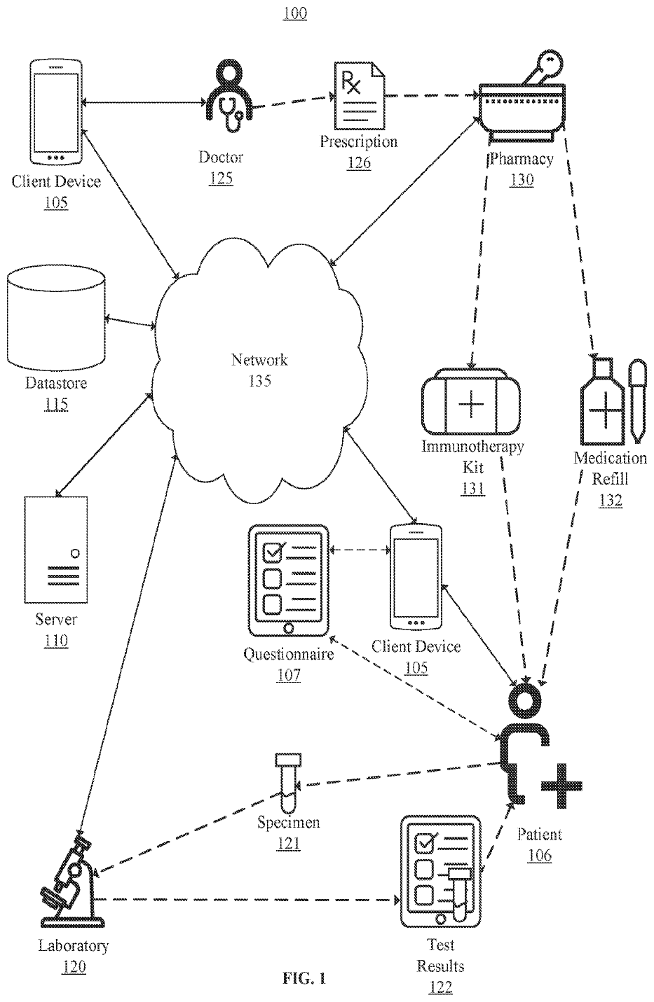

FIG. 1 is a diagram illustrating an exemplary framework for SLIT treatment determination and optimization 100, in which some embodiments may operate. The framework 100 may comprise client 105, server 110, datastore 115, laboratory 120, pharmacy 130 and network 135.

Patient 106 and doctor 125 may interact with the framework through client devices 105. Patient 106 may also receive a specimen collection kit through the mail or collected from a processing facility or physician's office. Patient 106 may collect specimen 121 and mail or otherwise deliver specimen 121 to laboratory 120. Laboratory 120 may process specimen 121 according to one or more assays and report test results 122 back to the user. The laboratory 120 may also share or otherwise send test result 122 to server 110, datastore 115, and to doctor 125 through network 135.

Patient 106 may also be provided with questionnaire 107. Questionnaire 107 may be completed by patient 106 on client device 105. Questionnaire may be transferred to server 110, datastore 115, and to doctor 125 through network 135.

Doctor 125 may communicate with patient 106 directly or through network 135. Doctor 125 may submit an order to laboratory 120 for one or more tests to be performed on sample 121. The order may be generated by client device 105 or server 110 and transferred to laboratory 120 over network 135. Doctor 125 may also review test result 122 and approve a prescription 126 generated by server 110. The prescription may be determined based on feedback from client 106, questionnaire 107 and test results 122. Prescription 126 may be transferred to pharmacy 130 over network 135. Pharmacy 130 may be configured to receive a treatment formulation, dosage and treatment schedule from server 110. Pharmacy 130 may then compound one or more SLIT treatments based on the information received from server 110. One or more immunotherapy kits 131 may be put together and mailed to patient 106. Medication refill 132 may be mailed to patient 106 at regular intervals based on their subscription.

Clients 105 may be any computing device capable of communicating over network 135. A client 105 may be a notebook computer, smartphone, personal digital assistant, desktop computer, tablet computer or other computing device. Server 110 may be one or more physical or virtual machines configured to communicate with the one or more clients 105, datastores 115, laboratory 120 and pharmacy 130. The one or more servers may be configured as a distributed computing infrastructure and processing of applications and other software may be carried out on the cloud.

Datastores 115 may communicate with one another over network 135. Datastores 115 may be any storage device capable of storing data for processing or as a result of processing information at the client 105, server 110, laboratory 120 and pharmacy 130. The datastores 115 may be a separate device or the same device as server 110. The datastore 115 may be located in the same location as that of server 110, or at separate locations.

Network 135 may be an intranet, internet, mesh, LTE, GSM, peer-to-peer or other communication network that allows the one or more servers 110 to communicate with the one or more clients 105, datastores 115, laboratory 120 and pharmacy 130.

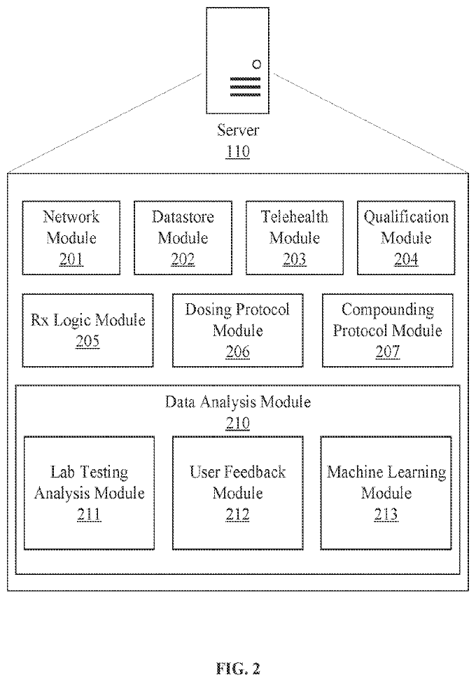

FIG. 2 is a diagram illustrating an exemplary server 110 in accordance with aspects of the present disclosure. Server 110 may comprise network module 201, datastore module 202, telehealth module 203, qualification module 204, Rx logic module 205, dosing protocol module 206, compounding protocol module 207 and data analysis module 210.

Network module 201 may transmit and receive data from other computing systems via a network. In some embodiments, the network module 201 may enable transmitting and receiving data from the Internet. Data received by the network module 201 may be used by the other modules. The modules may transmit data through the network module 201.

Datastore module 202 may be configured to store patient data for a plurality of patients. Datastore module 202 may be read from and written to by the other modules of server 110. Datastore module 202 may also store one or more datasets used for training by machine learning module 213.

Telehealth module 203 may be configured to facilitate communication between a physician and a patient. Through the telehealth module 203, the physician may provide consultation to a patient, review test results, diagnose conditions and issue prescriptions for the patient.

Qualification module 204 may be configured to determine the eligibility of a patient to receive treatment. In some embodiment, questionnaire and survey answers from a patient, patient's medical history and conditions that the patient has been diagnosed with may be used by the qualification module 204 to determine eligibility. For example, patients with a history of severe allergic reaction to immunotherapy or any ingredients in the immunotherapy may be disqualified from receiving treatment. In some embodiments, conditions such as pregnancy and breastfeeding status may disqualify a patient temporarily, and the qualification process may be repeated at a later time after the patient is no longer pregnant or breastfeeding. Other transient medical conditions may be treated in a similar manner.

Rx logic module 205 may be configured to take results of an allergy test and any meaningful phenotypic data collected during registration/activation or in follow-up surveys, and translates that into a prescription recommendation that a telehealth provider/physician may review and write a prescription for.

In some embodiments, the Rx logic module 205 may take into consideration a combination of factors and sources of information in the determination of a recommendation. In some embodiments, the combination of factors may include a list of allergens that may be testing for, a list of extracts available for treating one or more allergens, a mapping of allergen to extracts, regional pollination schedules, regional pollen activity calendars, a list of cross-reactive allergens and/or combination thereof. The regional pollination schedules and regional pollen activity calendars may be retrieved from a third party source, datastore 115 or datastore module 202. Retrieval of the regional pollination schedules and regional pollen activity calendars may be based at least in part on the location of the patient, travel habits of the patient, or other information that may be collected during registration or follow-up surveys. In some embodiments, a patient's historic location information may be analyzed to determine additional sources of allergens that may affect the patient. For example, a spatiotemporal mapping of allergen concentrations throughout a region may be compared to the location of the patient throughout the day to determine which allergens the patient comes into contact with, how often they come into contact with the allergens, concentration and duration of exposure to the allergens or other factors that may cause one or more allergens to cause more severe reactions than others.

Rx logic module 205 may further consider, in creating a prescription recommendation, the allergens tested for and the specific IgE levels of each allergen. The allergens tested for may be adjusted based on the location of the patient, patient's allergy history and patient survey. Survey information that may be used in generating the prescription recommendation may include a description of the allergic reactions experience by the patient, the severity of the reactions, a ranking of the allergens (severity or importance to the patient), the time-frame that patient has the highest symptoms, patient's city and state of residence, patient's city and state of employment and addresses of the patient's residence, place of employment. The time-frame that has the highest symptoms may be one or more months or entire seasons. In some embodiments, two adjacent months may be considered when analyzing the allergens present during the time of the patient's most severe symptoms.

Rx logic module 205 may be configured to analyze one or more patient test results and create a list of positive allergens based on IgE levels of each allergen. The IgE levels of each allergen may be compared against a threshold value, and the allergens with IgE levels above the threshold may then be added to the list of positive allergens in ranked order based on IgE levels (high-to-low). Allergen severity corresponds to IgE levels, and the higher the IgE level, the more severe the reaction/symptoms the patient may experience. If no allergens have an IgE level above the threshold the qualification module 204 may disqualify the patient and determine that the patient most likely is suffering from nonallergic rhinitis.

In some embodiments, the patient may also provide a list of allergy triggers when answering survey questions. The patient provided list may also be ranked by importance to the patient. The patient provided list may also include a time period for each trigger. The time period may be a single day or month or a range of days/months. When a single time is used, the two adjacent months may be associated with the trigger. The Rx logic module 205 may also generate a list of allergens based off of the location information collected from the patient. The regional pollination schedule may be used to identify allergens present in the patient's location at the times the patient suffers most from allergy symptoms. A list may be generated based off of the identified allergens for the specified time periods and location.

In some embodiments, the importance of the allergy trigger to the patient is weighted higher than the test results. When the highest ranked allergy trigger from the user created list has a corresponding IgE level above a threshold it may be classified as the first candidate allergy trigger to be treated (#1 allergen). If the IgE level does not exceed the threshold, the next highest user ranked allergy trigger may be analyzed in the same way. This may be repeated until the number of candidate allergy triggers to be treated are equal to the maximum number of extracts set for the prescription, or there are no more allergy triggers in the user created list or the patient result list.

In some embodiments, the selection of any additional allergen triggers after the selecting of the #1 allergen trigger may also be based on cross-reactivity between two or more allergens and/or extracts used to treat the allergen trigger. If the #1 allergen trigger is cross reactive with a second candidate allergen trigger, the second candidate allergen trigger may be ignored and the next highest allergen trigger becomes a new candidate allergen trigger. Each new allergen trigger may be analyzed to determine if there is cross reactivity between the new allergen trigger and any of the previously selected allergen trigger. In some embodiments, only non-cross-reactive allergy triggers are selected to be included in the generating of the prescription.

Dosing protocol module 206 may be configured to determine the SLIT immunotherapy dosing protocol. In some embodiments, the dosing protocol module 206 may analyze the patient's test results, patient's survey responses, the prescription, one or more allergen-extract mappings. Dosing volumes for the starter kit and maintenance kit, including any allowed variances in the protocol, may be determined based on the above analysis. One or more desired dilution for each extract may be determined based upon the dosing volumes, patient's test results, patient's survey responses, the prescription, one or more allergen-extract mappings. Dilutions for the starter kit and maintenance kit may be determined at the same time or at different times as one another. In some embodiments, one or more maintenance dosing volumes and dilutions may be determined based on the duration of the treatment plan and ongoing patient surveys taken by the patient during the course of treatment. In some embodiments, a survey may be taken after each completed dilution period, at regular intervals, randomly or after adverse events. In some embodiments, the duration of the treatment plan and the duration at each dilution level may be determined at the beginning of treatment. In some embodiments, the duration of the treatment plan and the duration at each dilution level may be determined at regular intervals or after receiving additional patient information, such as the ongoing surveys, reports of adverse events and blood test results of samples taken during the course of treatment.

In some embodiments, dosing protocol module 206 may be configured to determine the number of doses per day for a given patient. The determination of the number of doses may be based upon the patient's ability to adhere to a recommended or optimal dosing schedule. In some embodiments, a suboptimal dosing schedule may be adopted to make it more convenient for the patient. For example, if the optimal dosing schedule is for the patient to take the treatment three times a day with each treatment being separated by six hours, the patient may have difficulties administering the doses at those times due to their work/life schedule or just forgetting. In these cases, the dosing schedule may be adjusted or determined before start of treatment to be taken once a day to reduce the burden on the patient. In some embodiments, when a dosing schedule prescribes a single dose, the dosing protocol module 206 may adjust the dosing schedule to increase the number of doses during the day but at lower dilutions/concentrations. For example, if a patient has unpleasant side effects at a higher single dosage, the spacing apart of lower dosages may be tolerated by the patient better.

In some embodiments, dosing protocol module 206 may be configured to generate abnormal dosing schedules based on one or more events that occur during the course of treatment. For example, dosing schedules for missed doses on build up, missed doses on maintenance, severe reaction at the beginning of a new dose dilution and severe reaction in the middle of any dilution or maintenance phase may be uniquely generated for each patient, generated for groups of patients or generated for all patients.

Compounding protocol module 207 may be configured to determine the mixing of extracts to create the treatment solutions based at least partly on the prescription generated for the patient. The compounding may also be at least partly based on volumes required during each phase of treatment (build up or maintenance), compounding supplies and inventory on hand at the compounding pharmacy, extract suppliers and rules around using primary and secondary extract suppliers, mixing guidelines, and substitute guidelines. In some embodiments, compounding protocol module 207 may be configured to track inventory of extracts, as well as determine and enforce minimum quantities on hand based on forecasts. The forecasts may be based upon a safety stock quantity plus a days on hand quantity. The quantities may be based upon patient subscriptions and the forecast of future need based on each patient and their needs throughout their course of treatment.

Data analysis module 210 may comprise lab testing analysis module 211, user feedback module 212 and machine learning module 213.

Lab testing analysis module 211 may be configured to receive and analyze test results received from laboratory 120. User feedback module 212 may be configured to analyze patient input received from patient intake surveys, questionnaires, and ongoing surveys. Machine learning module 213 may be configured to receive raw, preprocessed, postprocessed or other data relating to the analysis of lab test results and patient feedback from the lab testing analysis module 211 and user feedback module 212. Machine learning module 213 may include one or more machine learning models for analyzing, classifying, generating, evolving, learning and predicting of allergen triggers, severity of triggers, cross-reactivity of allergen triggers, ranking of allergen triggers, selection of allergen triggers to be treated, treatment plan schedules, dosing protocols, dosing volumes, dosing dilution, dosing schedule, compounding protocols, inventory tracking, inventory ordering, qualification of the patient, adverse events or combination thereof.

In some embodiments, machine learning module 213 may be configured to train or retrain one or more machine learning models at regular intervals based off of data collected on patients during their course of treatment. In some embodiments, training and retraining may use a combination of synthetically generated testing data along with actual patient data.

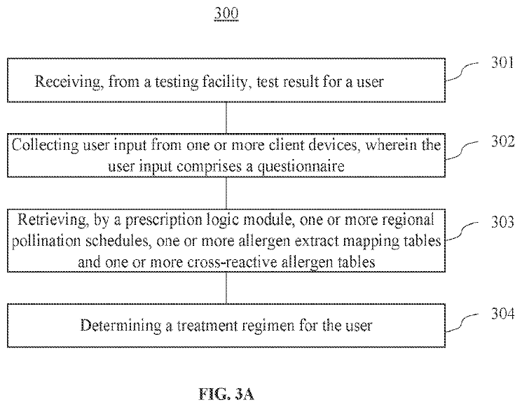

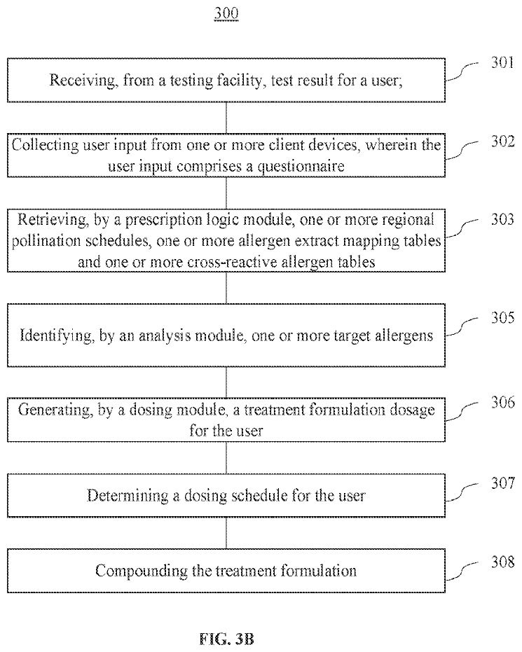

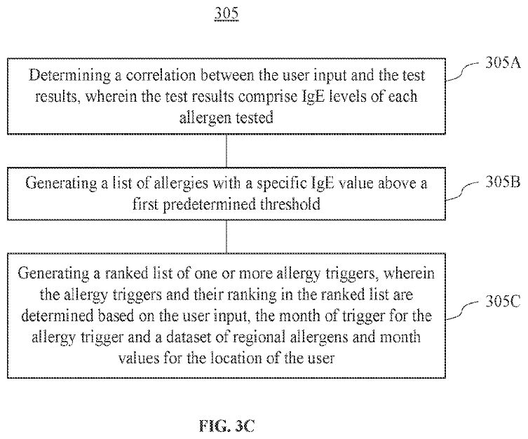

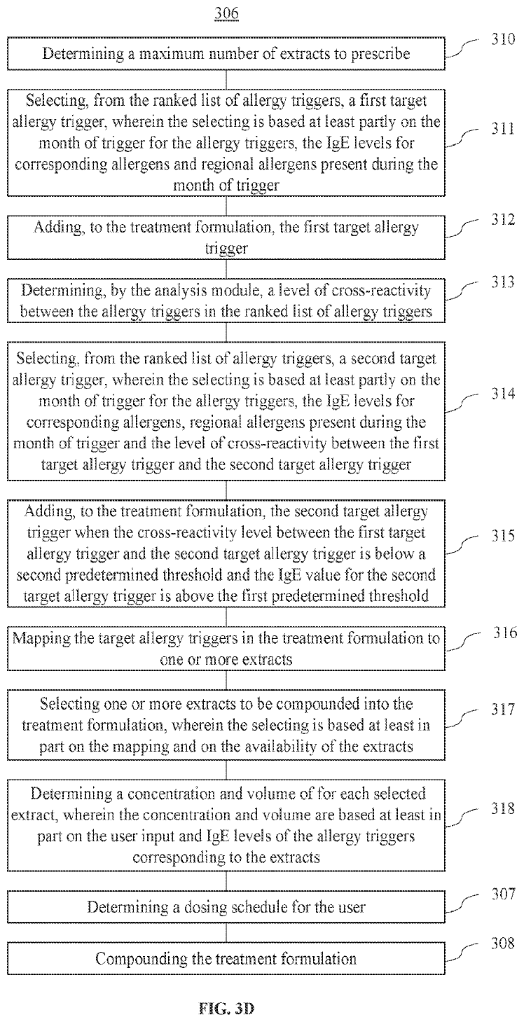

FIG. 3A is a flow chart illustrating an exemplary method 300A that may be performed in accordance with some embodiments.

At step 301, the system is configured to receive, from a testing facility, test result for a user.

At step 302, the system is configured to collect user input from one or more client devices, wherein the user input comprises a questionnaire.

At step 303, the system is configured to retrieve, by a prescription logic module, one or more regional pollination schedules, one or more allergen extract mapping tables and one or more cross-reactive allergen tables.

At step 304, the system is configured to determine a treatment regimen for the user.

FIG. 3B is a flow chart illustrating an exemplary method 300 that may be performed in accordance with some embodiments. Steps 301-303 may be the same or similar to that of steps 301-303 of FIG. 3A.

At step 305, the system is configured to identify, by an analysis module, one or more target allergens.

At step 306, the system is configured to generate, by a dosing module, a treatment formulation dosage for the user.

At step 307, the system is configured to determine a dosing schedule for the

At step 308, the system is configured to compound the treatment formulation.

FIG. 3C is a flow chart illustrating the exemplary process comprised in step 305 of FIG. 3B in accordance with some embodiments.

At step 305A, the system is configured to determine a correlation between the user input and the test results, wherein the test results comprise IgE levels of each allergen tested

At step 305B, the system is configured to generate a list of allergies with a specific IgE value above a first predetermined threshold.

At step 305C, the system is configured to generate a ranked list of one or more allergy triggers, wherein the allergy triggers and their ranking in the ranked list are determined based on the user input, the month of trigger for the allergy trigger and a dataset of regional allergens and month values for the location of the user.

FIG. 3D is a flow chart illustrating the exemplary process comprised in step 306 of FIG. 3B in accordance with some embodiments.

At step 310, the system is configured to determine a maximum number of extracts to prescribe.

At step 311, the system is configured to select, from the ranked list of allergy triggers, a first target allergy trigger, wherein the selecting is based at least partly on the month of trigger for the allergy triggers, the IgE levels for corresponding allergens and regional allergens present during the month of trigger.

At step 312, the system is configured to add, to the treatment formulation, the first target allergy trigger.

At step 313, the system is configured to determine, by the analysis module, a level of cross-reactivity between the allergy triggers in the ranked list of allergy triggers.

At step 314, the system is configured to select, from the ranked list of allergy triggers, a second target allergy trigger, wherein the selecting is based at least partly on the month of trigger for the allergy triggers, the IgE levels for corresponding allergens, regional allergens present during the month of trigger and the level of cross-reactivity between the first target allergy trigger and the second target allergy trigger.

At step 315, the system is configured to add, to the treatment formulation, the second target allergy trigger when the cross-reactivity level between the first target allergy trigger and the second target allergy trigger is below a second predetermined threshold and the IgE value for the second target allergy trigger is above the first predetermined threshold.

At step 316, the system is configured to map the target allergy triggers in the treatment formulation to one or more extracts.

At step 317, the system is configured to select one or more extracts to be compounded into the treatment formulation, wherein the selection is based on the mapping of the extracts.

At step 318, the system is configured to determine a concentration and volume of for each selected extract, wherein the concentration and volume are based at least in part on the user input and IgE levels of the allergy triggers corresponding to the extracts.

Steps 307 and 308 are the same as in FIG. 3B

FIG. 3D is a flow chart illustrating an exemplary method 320 that may be performed in accordance with some embodiments.

At step 321, the patient requests test kit. At step 322, the test kit is shipped to the patient. At step 323, the patient registers an account on server 110 through a web based user interface. At step 324, the patient registers the test kit and the test kit is associated with the patient's account. At step 325, the patient takes a survey or questionnaire through the user interface. At step 326, the system establishes a physician/patient relationship and requests physician authorization for the test. At step 327, the patient collects the specimen and returns the specimen. The specimen is returned through a shipping service or the mail. At step 328, the lab receives the specimen. At step 329, the lab processes the specimen. At step 330, the results from the specimen processing are shared with the patient, physician and server 110. At step 331, the system is configured to qualify the results of the test and determine if the patient is qualified for the treatment regimen. At step 332, the patient has a synchronous or asynchronous visit with a physician to review the results of the blood test as well as answers from the survey taken by the patient in step 325. At step 333, the physician issues a prescription and sends the prescription to the compounding pharmacy. At step 334, the patient subscribes to a recurring-monthly, quarterly, yearly-service to receive treatment for their allergies. At step 335, the pharmacy compounds the treatment based on the prescription received from the physician. At step 336, the compounded treatment is shipped to the patient. At step 337, the patient answers an ongoing survey to track the progress of the patients symptoms. Steps 335, 336 and 337 are repeated until the prescription has expired or based on the ongoing survey, a physician consultation is required for a review and/or new issuance of a prescription. If a physician consultation is required, the process returns to step 332 and repeats.

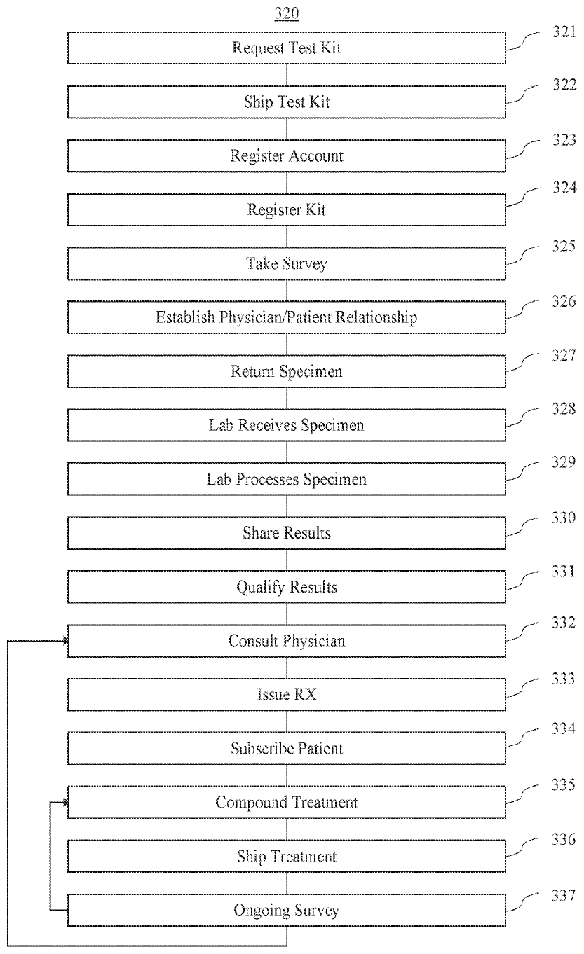

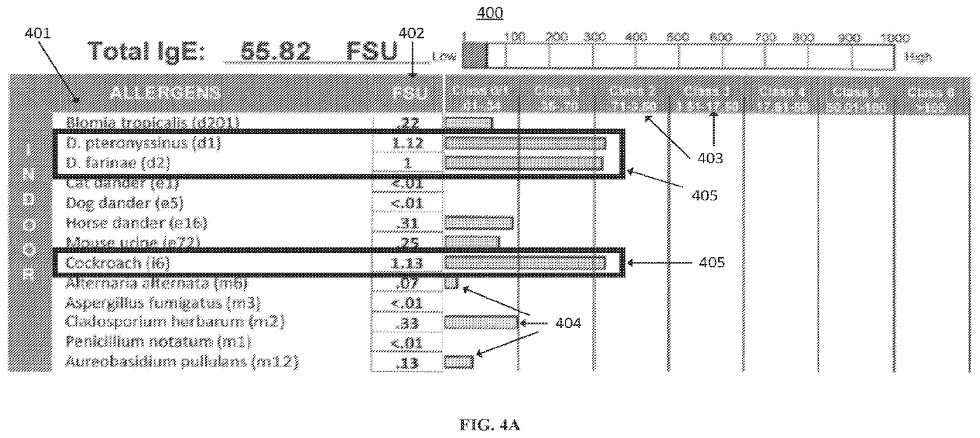

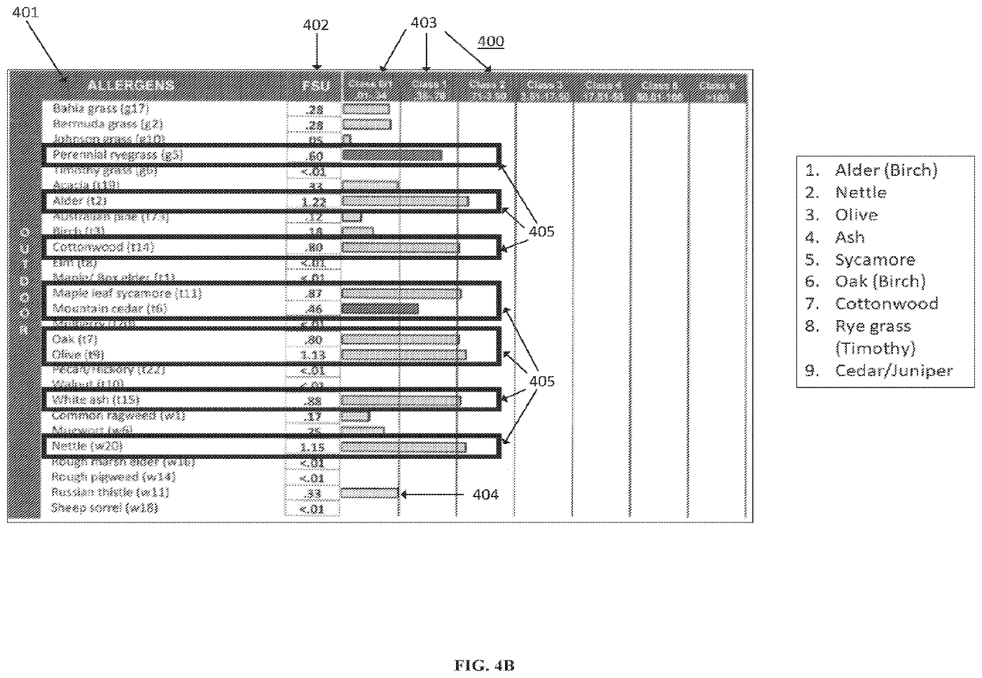

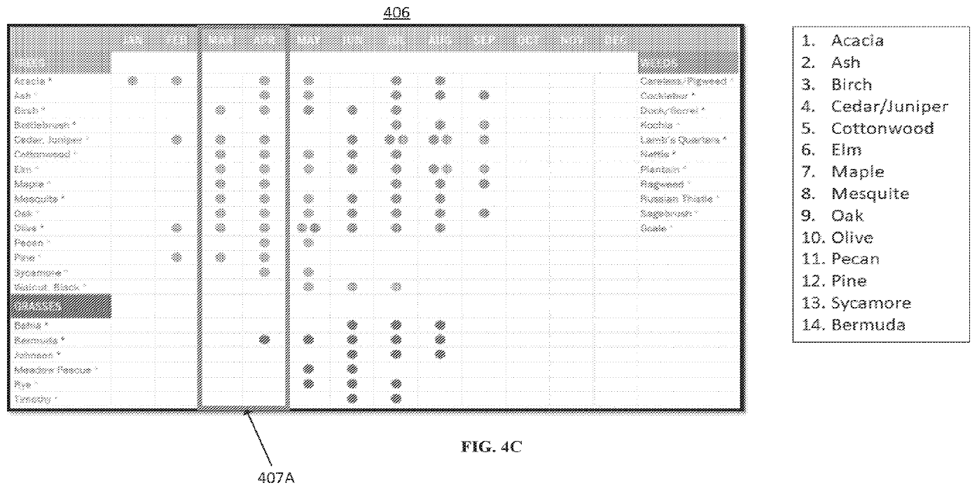

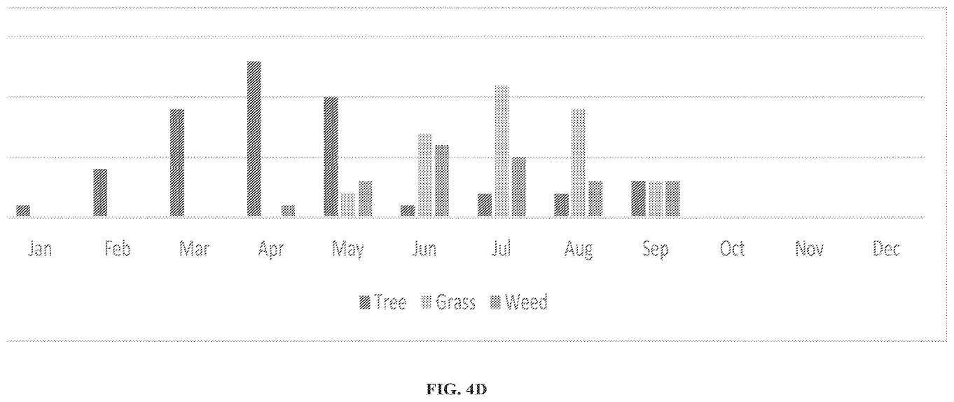

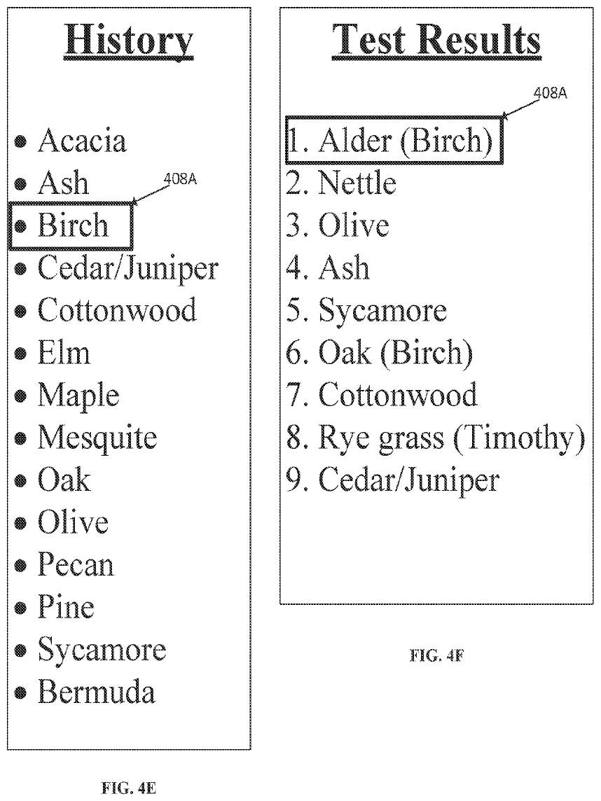

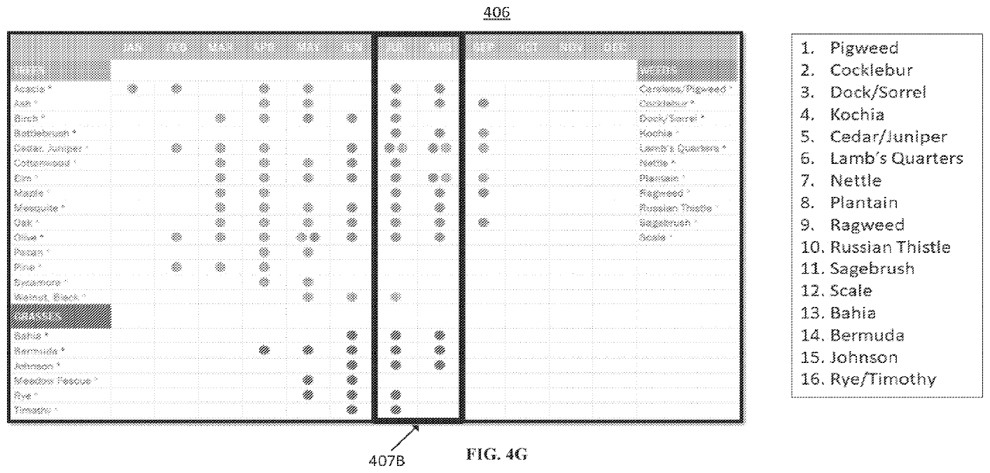

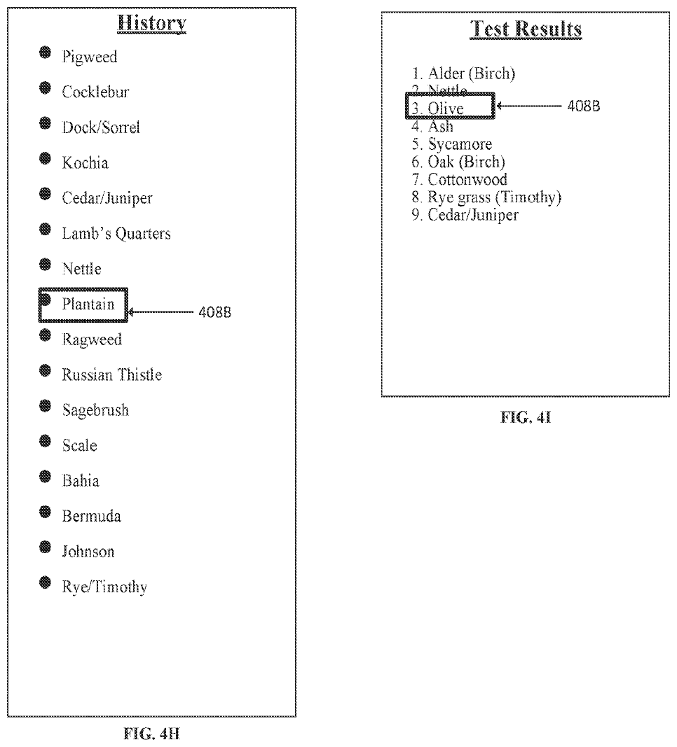

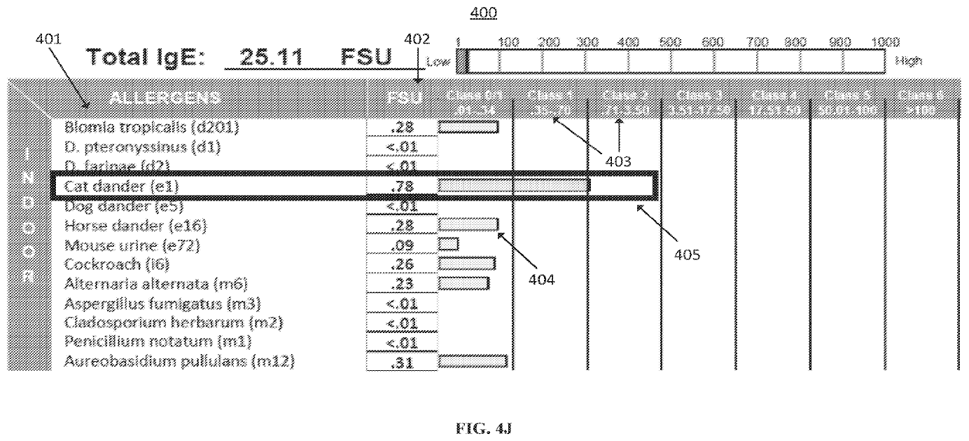

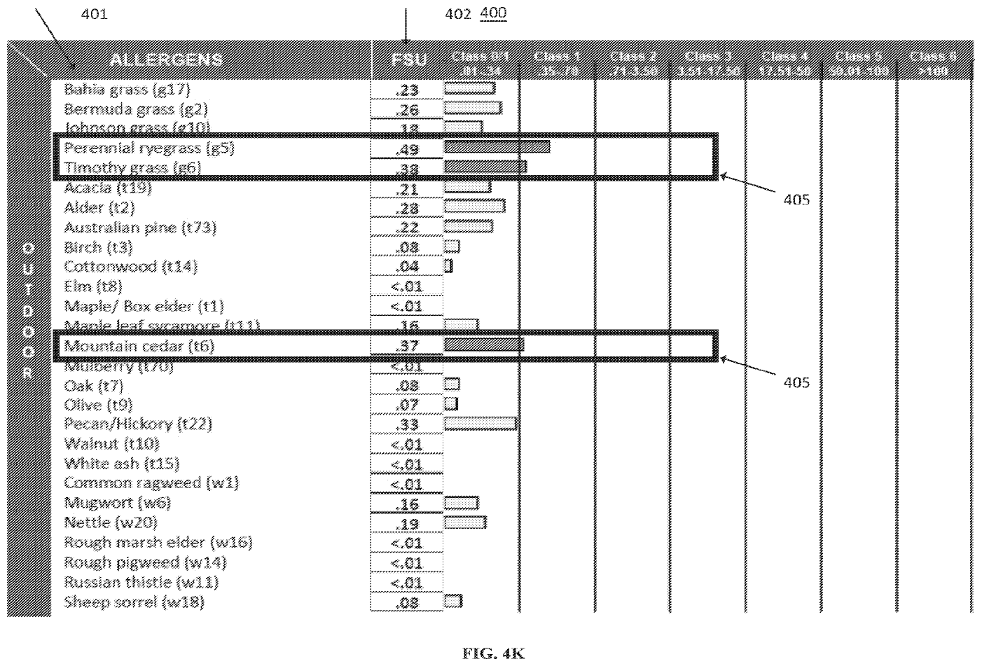

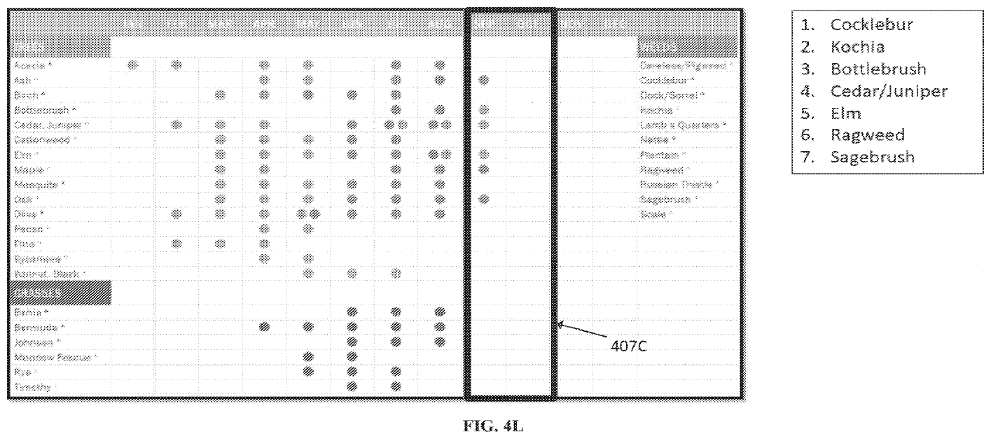

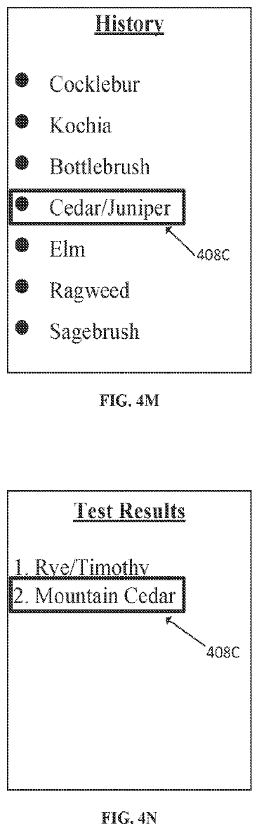

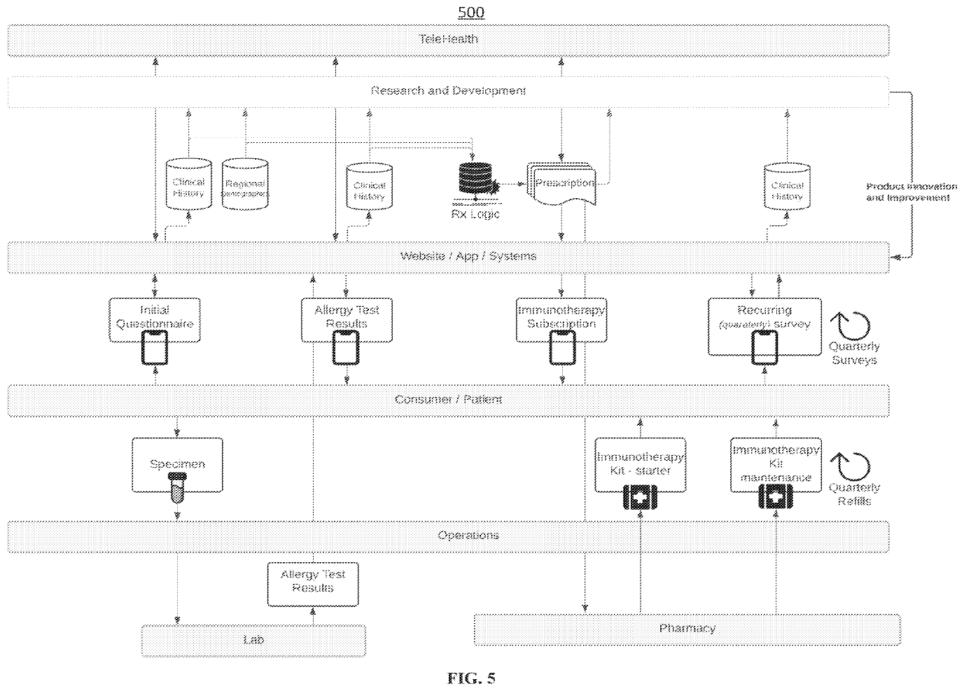

FIGS. 4A, 4B, 4J and 4K show exemplary allergy test results 400 in accordance with some embodiments. FIGS. 4A and 4B are test results belonging to a first patient, and FIGS. 4J and 4K are test results belonging to a second patient.

Allergy test results 400 may comprise list of allergens 401, IgE values for individual allergens 402 and classification of allergens 403. Allergens may be classified based on one or more predetermined thresholds. Item 404 is an example of an allergen that is below the lowest threshold and not of any significance. Items 405 are examples of allergens that are above at least one threshold value. These allergens may be used in the development of a treatment plan for the patient.

FIGS. 4C, 4D, 4G, 4L show exemplary regional allergen pollination schedules 406 in accordance with some embodiments.

Trigger period 407A, 407B and 407C show the time of year when the patient experiences allergy symptoms. The allergens present at these times in the patients region are used to generate a list of possible allergens to include in treatment.

FIGS. 4E, 4H and 4M show exemplary patient allergy histories and FIGS. 4F, 4I and 4N show exemplary ranked lists of allergens in accordance with some embodiments.

FIGS. 4E, 4F, 4H and 4I are allergy histories and ranked lists of allergens for a first patient. FIGS. 4M and 4N are an allergy history and ranked list of allergens for a second patient.

FIG. 4E shows a list of allergies present during trigger period 407A (March and April) from FIG. 4C. FIGS. 4E and 4F show a selection of a first trigger allergen 408A that the patient suffers from during trigger period 407A.

FIG. 4H shows a list of allergies present during trigger period 407B (July and August) from FIG. 4G. FIGS. 4H and 4I show a selection of a second trigger allergen 408B that the patient suffers from during trigger period 407B.

FIG. 4M shows a list of allergies present during trigger period 407C (September and October) from FIG. 4L. FIGS. 4M and 4N show a selection of a first trigger allergen 408C that a second patient suffers from during trigger period 407B.

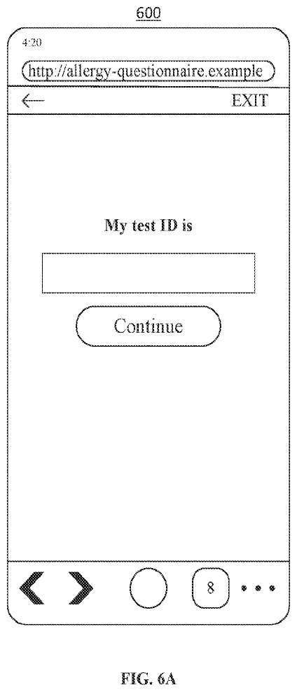

FIG. 5 is an exemplary framework diagram in accordance with some embodiments. FIG. 5 shows the main actors in the operation of the SLIT treatment determination and optimization framework (TeleHealth, Website/App/Systems, Patient, Operations, Lab and Pharmacy). There may be more or fewer actors in the SLIT treatment determination and optimization framework than that shown in FIG. 5. FIG. 5 also shows the connection between the actors and objects/events that take place during the entire treatment process.

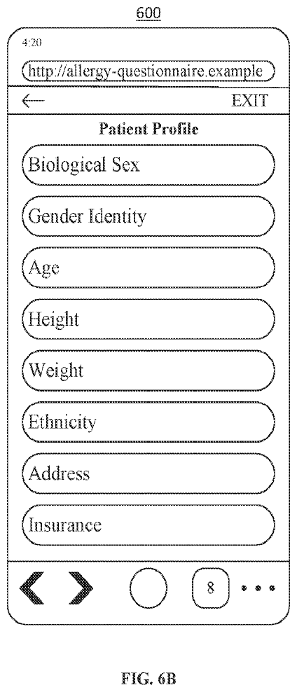

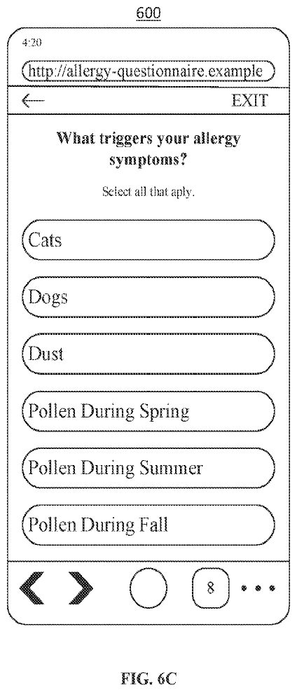

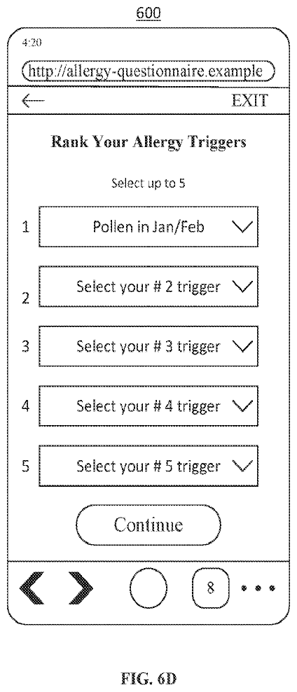

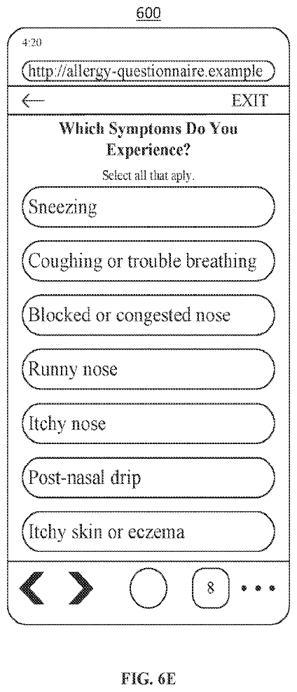

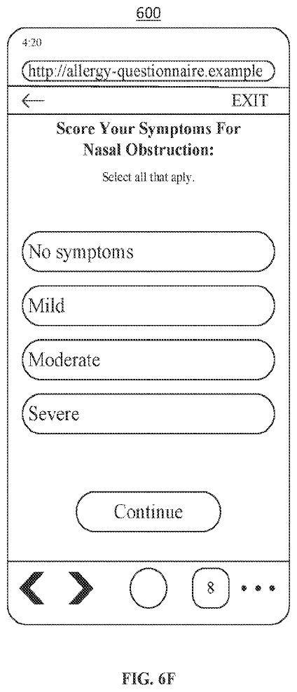

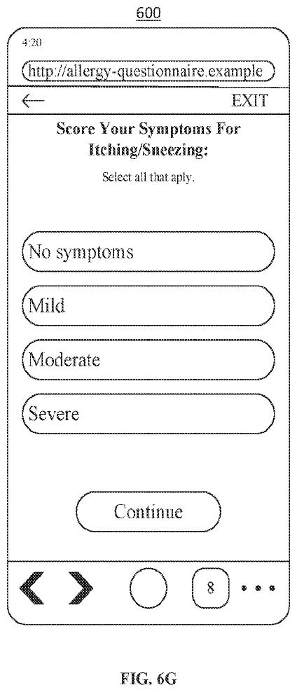

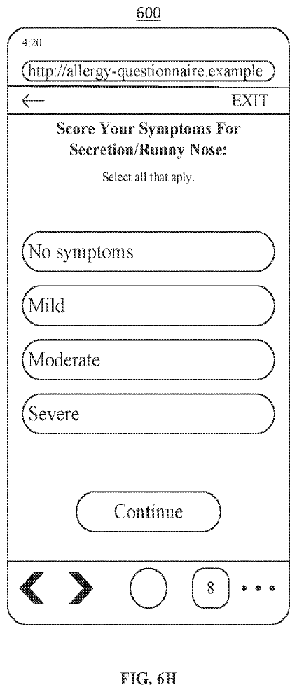

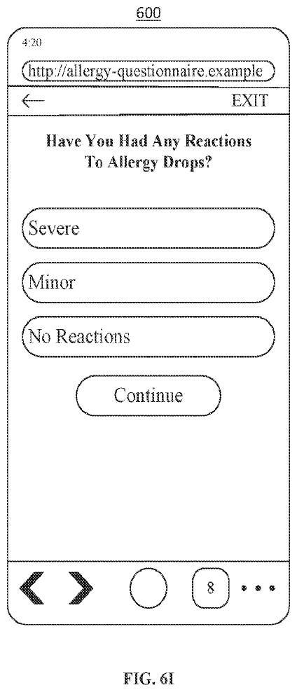

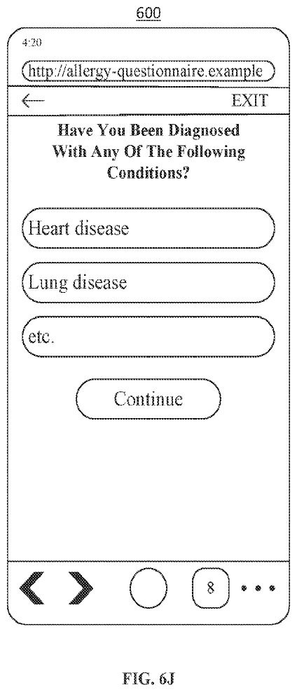

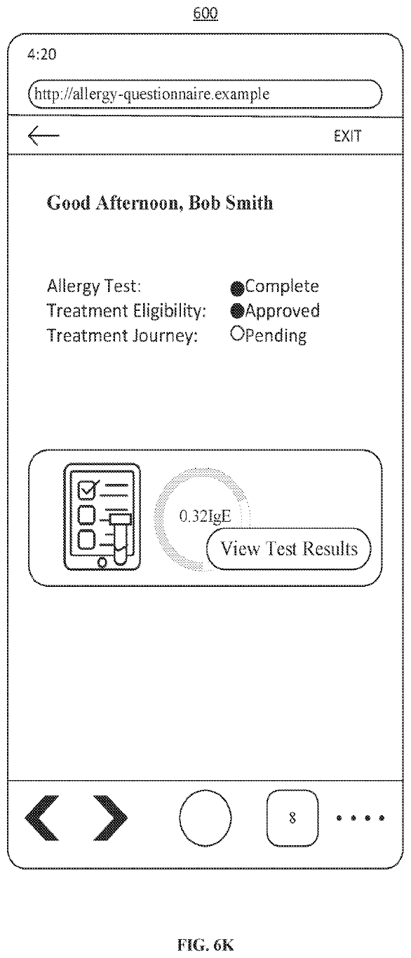

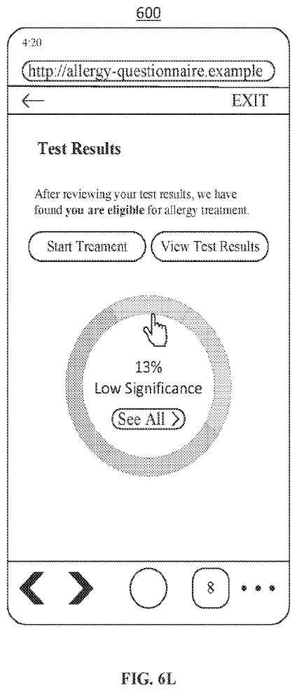

FIGS. 6A-6L show an exemplary user interface 600 in accordance with some embodiments. In FIG. 6A, the patient is prompted to enter their test ID so that it can be registered and associated with the patient's account. In FIG. 6B, the patient is prompted to enter personal information in the patient profile. In FIG. 6C, the patient may select one or more allergy triggers. The selected allergy triggers may be used as a patient history. In FIG. 6D, the patient is asked to rank their allergy triggers. In the example, the patient can rank up to five triggers. In some embodiments, less or more triggers may be ranked by the patient. In FIG. 6E, the patient selects the symptoms that they experience when suffering from allergies. In FIG. 6F-6H, the patient may score the symptoms that were selected in FIG. 6E. In FIG. 61, the patient may be asked to indicate if they have had any reactions to allergy drops or allergy shots in the past. In FIG. 6J, the patient selects, from a list of conditions, the conditions that they have been diagnosed with. In FIG. 6K, the user interface may be configured to display a list of steps in the treatment process and the status of each step. For example, in FIG. 6K, the patient has completed the allergy test, their treatment eligibility has been approved and the treatment journey is pending. The patient may select to view the results of the allergy test at this point. In FIG. 6L, the patient may be shown a chart or diagram representing the results of the allergy test. In some embodiments, the results may be classified into four categories. These categories may include no significance, low significance, moderate significance and high significance. The classification of each of the allergens tested for may be based on the IgE value for that specific allergen. The percentage of the allergens tested for that are classified into each of the categories may be used to generate a visual representation of the test results.

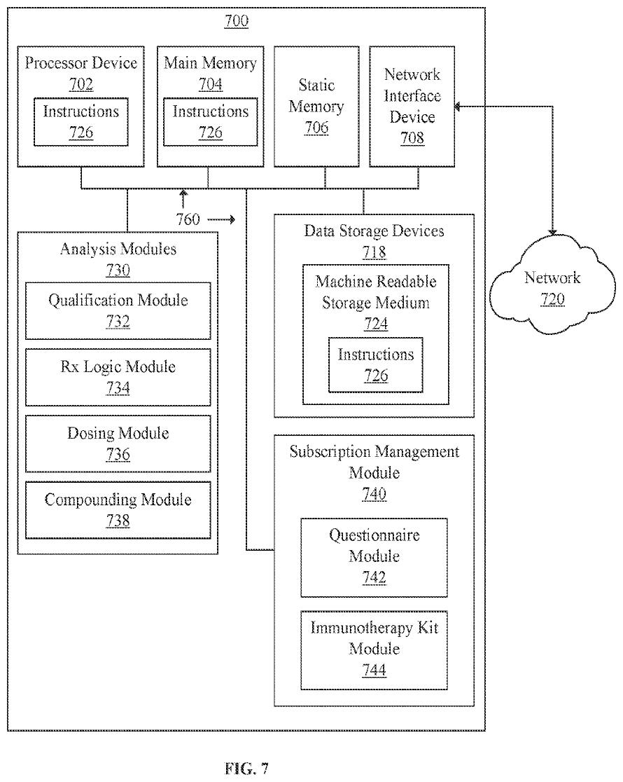

FIG. 7 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, an ad-hoc network, a mesh network, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 700 includes a processing device 702, a main memory 704 (e.g., read-only memory (ROM), flash memory, dynamic random access memory (DRAM) such as synchronous DRAM (SDRAM) or Rambus DRAM (RDRAM), etc.), a static memory 706 (e.g., flash memory, static random access memory (SRAM), etc.), and a data storage device 718, which communicate with each other via a bus 760.

Processing device 702 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute instructions 726 for performing the operations and steps discussed herein.

The computer system 700 may further include a network interface device 708 to communicate over the network 720. The computer system 700 also may include sensor array 710. Sensor array 710 may comprise a camera sensor 712, infrared sensor 714 and depth sensor 716.

The data storage device 718 may include a machine-readable storage medium 724 (also known as a computer-readable medium) on which is stored one or more sets of instructions or software 726 embodying any one or more of the methodologies or functions described herein. The instructions 726 may also reside, completely or at least partially, within the main memory 704 and/or within the processing device 702 during execution thereof by the computer system 700, the main memory 704 and the processing device 702 also constituting machine-readable storage media.

Analysis Modules 730 may be the same or similar to that of data analysis module 205 of FIG. 2. Qualification Module 732 may be the same or similar to that of qualification module 204 of FIG. 2. Rx Logic Module 734 may be the same or similar to that of Rx logic module 205 of FIG. 2. Dosing Module 736 may be the same or similar to that of dosing protocol module 206 of FIG. 2. Compounding Module 738 may be the same or similar to that of compounding protocol module 207 of FIG. 2.

Subscription Management Module 740 may comprise questionnaire module 742 and immunotherapy kit module 744. Subscription Management Module 740 may be configured to use one or more machine learning models of the machine learning module 213 to analyze data received from the qualification module 732, Rx logic module 734, dosing module compounding module 738, questionnaire module 742 and immunotherapy kit module 744.

Questionnaire Module 742 may be the same or similar to that of user feedback module 212 of FIG. 2.

Immunotherapy kit module 744 may be configured to determine the contents of the immunotherapy kit that is to be shipped to the patient. The immunotherapy kit module 744 may also coordinate retrieval of the compounded immunotherapy solutions, the packing of the compounded immunotherapy solutions, the inclusion of dosing schedules within the immunotherapy kit, the shipment of the immunotherapy kit and tracking of the immunotherapy kit after shipment.

In one implementation, the instructions 726 include instructions to implement functionality corresponding to the components of a device to perform the disclosure herein. While the machine-readable storage medium 724 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer implemented method for determining and optimizing an individualized treatment regimen for a patient, the method comprising:
receiving, from a testing facility, a first test result for a user;
collecting user input from one or more client devices, wherein the user input comprises a questionnaire;
determining a treatment regimen for the user based at least in part on the user input and the first test result, wherein the determining comprises:
generating, by a dosing module, a treatment formulation based at least in part on the first test result and the user input; and
determining, by the dosing module, a dosing schedule for the user based at least in part on the treatment formulation;
issuing, by a physician, a first prescription based on the treatment formulation and sending the first prescription to a first compounding pharmacy;
compounding, at the first compounding pharmacy, a first compounded treatment based on the first prescription;
shipping, from the first compounding pharmacy to the user, the first compounded treatment; and
starting, by the user, the treatment regimen using the first compounded treatment and wherein the first compounded treatment is administered based on the determined dosing schedule.

2. The computer implemented method of claim 1, wherein determining the treatment regimen further comprises:
retrieving, by a prescription logic module, one or more regional pollination schedules, one or more allergen extract mapping tables and one or more cross-reactive allergen tables, wherein the retrieving is based at least in part on the user input, the first test result and a location of the user;

3. The computer implemented method of claim 2, wherein determining the treatment regimen further comprises:
identifying, by an analysis module, one or more target allergens, the identifying comprising:
determining a correlation between the user input and the first test results, wherein the first test result comprises IgE levels of each allergen tested;
generating a list of allergies with a specific IgE value above a first predetermined threshold; and
generating a ranked list of one or more allergy triggers, wherein the allergy triggers and their ranking in the ranked list are determined based on the user input, a month of trigger for the allergy trigger and the one or more retrieved regional pollination schedules.

4. The computer implemented method of claim 3, wherein generating the treatment formulation further comprises:
selecting, from the ranked list of allergy triggers, a first target allergy trigger;
adding, to the treatment formulation, the first target allergy trigger;
selecting, from the ranked list of allergy triggers, a second target allergy trigger;
adding, to the treatment formulation, the second target allergy trigger;
mapping the target allergy triggers in the treatment formulation to one or more extracts;
selecting one or more extracts to be compounded into the treatment formulation; and
determining a concentration and volume of for each selected extract.

5. The computer implemented method of claim 4, wherein selecting the first target allergy trigger is based at least partly on a month of trigger for the allergy triggers, the IgE levels for corresponding allergens and regional allergens present during the month of trigger;

6. The computer implemented method of claim 5, wherein generating the treatment formulation further comprises:
determining, by the analysis module, a level of cross-reactivity between the allergy triggers in the ranked list of allergy triggers.

7. The computer implemented method of claim 6, wherein selecting the second target allergy trigger is based at least partly on the month of trigger for the allergy triggers, the IgE levels for corresponding allergens, regional allergens present during the month of trigger and the level of cross-reactivity between the first target allergy trigger and the second target allergy trigger.

8. The computer implemented method of claim 7, wherein second target allergy trigger is added to the treatment formulation if the cross-reactivity level between the first target allergy trigger and the second target allergy trigger is below a second predetermined threshold and the IgE value for the second target allergy trigger is above the first predetermined threshold;

9. The computer implemented method of claim 8, wherein generating the treatment formulation further comprises:
mapping the target allergy triggers in the treatment formulation to one or more extracts;
selecting one or more extracts to be added to the first prescription for the treatment formulation, wherein the selecting is based at least in part on the mapping and on availability of the extracts; and
determining a concentration and volume of for each selected extract, wherein the concentration and volume are based at least in part on the user input and IgE levels of the allergy triggers corresponding to the extracts;

10. A platform for determining and optimizing an individualized treatment regimen for a patient, the platform comprising:
one or more servers;
one or more client devices;
one or more fulfillment centers;
one or more laboratory testing facilities;
one or more compounding pharmacies;
one or more physicians;
wherein the platform is further configured for:
receiving, at the one or more servers, a request from the patient, wherein the request comprises an order for a testing kit;
shipping, from a fulfillment center, a first testing kit;
creating a patient account, wherein the creating comprises:
receiving, at the one or more servers, patient information collected during a registration process, wherein the registration process includes collecting patient input from the patient, wherein the input comprises answers to a questionnaire or survey and a first testing kit ID; and
associating the first testing kit with the patient account based on the first testing kit ID;
establishing, through a telehealth system, a relationship between the patient and a physician;
collecting, by the first testing kit, a first specimen from the patient;
shipping, by the patient to a first laboratory testing facility, the first specimen;
receiving, by the one or more servers, first test results for the first specimen, wherein the first test results are determined by processing the first specimen at the first testing facility;
qualifying, by the one or more servers, an eligibility of the patient for sublingual immunotherapy treatment;
determining, by an analysis module operating on the one or more servers, a first treatment regimen for the patient;
initiating a consultation, for the patient, with the physician;
issuing, by the physician, a prescription, wherein the prescription is based on the determined first treatment regimen and wherein the prescription is sent to a first compounding pharmacy;
receiving, from the patient, a subscription registration, wherein the subscription comprises providing the patient with compounded treatments at regular intervals;
compounding, at the first compounding pharmacy, a first compounded treatment based on the prescription issued by the physician;
shipping, to the patient from the first compounding pharmacy, the first compounded treatment; and
administering, by the patient, the first compounded treatment based on the determined first treatment regimen.

11. The platform of claim 10, wherein determining the treatment regimen further comprises:
retrieving, by a prescription logic module, one or more regional pollination schedules, one or more allergen extract mapping tables and one or more cross-reactive allergen tables, wherein the retrieving is based at least in part on the patient input, the first test results and a location of the patient;

12. The platform of claim 11, wherein determining the treatment regimen further comprises:
identifying, by the analysis module, one or more target allergens, the identifying comprising:
determining a correlation between the patient input and the first test results, wherein the first test results comprises IgE levels of each allergen tested;
generating a list of allergies with a specific IgE value above a first predetermined threshold; and
generating a ranked list of one or more allergy triggers, wherein the allergy triggers and their ranking in the ranked list are determined based on the patient input, a month of trigger for the allergy trigger and the one or more retrieved regional pollination schedules.

13. The platform of claim 12, wherein generating the treatment formulation further comprises:
selecting, from the ranked list of allergy triggers, a first target allergy trigger;
adding, to the treatment formulation, the first target allergy trigger,
selecting, from the ranked list of allergy triggers, a second target allergy trigger;
adding, to the treatment formulation, the second target allergy trigger;
mapping the target allergy triggers in the treatment formulation to one or more extracts;
selecting one or more extracts to be compounded into the treatment formulation; and
determining a concentration and volume of for each selected extract.

14. The platform of claim 13, wherein selecting the first target allergy trigger is based at least partly on a month of trigger for the allergy triggers, the IgE levels for corresponding allergens and regional allergens present during the month of trigger;

15. The platform of claim 14, wherein generating the treatment formulation further comprises:
determining, by the analysis module, a level of cross-reactivity between the allergy triggers in the ranked list of allergy triggers.

16. The platform of claim 15, wherein selecting the second target allergy trigger is based at least partly on the month of trigger for the allergy triggers, the IgE levels for corresponding allergens, regional allergens present during the month of trigger and the level of cross-reactivity between the first target allergy trigger and the second target allergy trigger.

17. The platform of claim 16, wherein second target allergy trigger is added to the treatment formulation if the cross-reactivity level between the first target allergy trigger and the second target allergy trigger is below a second predetermined threshold and the IgE value for the second target allergy trigger is above the first predetermined threshold;

18. The platform of claim 17, wherein generating the treatment formulation further comprises:
mapping the target allergy triggers in the treatment formulation to one or more extracts;
selecting one or more extracts to be added to the prescription for the treatment formulation, wherein the selecting is based at least in part on the mapping and on availability of the extracts; and
determining a concentration and volume for each selected extract, wherein the concentration and volume are based at least in part on the patient input and IgE levels of the allergy triggers corresponding to the extracts.
